(19) United States
(12) Patent Application Publication
(10) Pub. No.: US 2008/0065411 A1
(43) Pub. Date: Mar. 13, 2008

(54) Method and system for developing a personalized medicine business plan

(75) Inventors: Peter Keeling, Belfast (GB); Dave Smart, Northern Ireland (GB)

Correspondence Address: FOLEY AND LARDNER LLP;SUITE 500, 3000 K STREET NW, WASHINGTON, DC, 20007, US

(73) Assignee: Diaceutics

(21) Appl. No.: 11/517,591

(22) Filed: Sep. 8, 2006

(51) Int. Cl. G06Q 10/00; A61B 5/00

(52) U.S. Cl. 705/2; 600/300

(57) ABSTRACT

A method of developing a personalised medicine business plan comprising the steps of:
obtaining a plurality of variables associated with an intended personalised medicine business plan;
generating a predicted revenue from the plurality of variables associated with the intended personalised medicine business plan;
collating the plurality of variables associated with the intended personalised medicine business plan and the predicted revenue to generate a hypothetical business scenario;
creating an archive of scenarios comprising data relating to personalised medicine, said data being acquired from real-world case studies;
comparing each of the scenarios in the archive with the hypothetical business scenario to identify a first archived scenario that most closely matches at least some of the variables of the hypothetical business scenario; and
extracting information from the first archived scenario, said information being used to justify the intended personalised medicine business plan and provide one or more guidance parameters on how to implement the intended personalised medicine business plan and thereby develop the intended personalised medicine business plan.

### FIELD OF THE INVENTION

The present invention relates to a method and system for developing a personalised medicine business plan.

### BACKGROUND OF THE INVENTION

Personalised medicine refers to the use of a test (or diagnostic) to target a drug (or therapy) at patients that are most likely to benefit therefrom, or to identify patients who may be at risk of harm from said therapy. The terms “theranostic” and “targeted therapy” respectively refer to the test and drug aspects of a personalised medicine (e.g. a theranostic is a test that is closely aligned (or specifically aligned) with a particular drug). Recent years have seen growing support for the use of personalised medicine to overcome limitations with the traditional “blockbuster” pharmaceutical business model (wherein pharmaceutical companies build their operations around a few products that produce the bulk of their revenues).

The “blockbuster” pharmaceutical business model is based on an assumption that a single compound can effectively treat most or all patients with a particular condition. However, recent studies have shown that more than 50% of patients do not respond to drugs used across many therapeutic categories (Trends in Molecular Medicine 7(5): 201-204, 2001). This limitation impacts on the process of drug approval, as clinical trials must be large enough to show the efficacy of a drug, even if a large proportion of trial participants are non-responsive to the drug. However, such large trials are lengthy and expensive. Furthermore, the non-responsiveness of a large proportion of trial participants to a drug may be interpreted by a regulatory authority as an indication that the drug is more generally ineffective; thereby leading to the rejection of an otherwise effective drug. Further problems arise when drugs are approved and released into the wider marketplace, whereupon adverse effects that did not appear in clinical trials come to light. A recent study has shown that 2.2 million Americans per year suffer adverse reactions to prescription drugs, leading to 100,000 deaths. This is a serious problem for pharmaceutical companies because of the enormous costs of product recalls and litigation.

Recent advances in the understanding of the molecular pathways of disease have enabled new diagnostic tools to be developed to predict and monitor a patient's response to a drug. These diagnostic tools enable the identification of patients that are most likely to respond to a drug with minimal side effects and those that are most likely to suffer serious side effects from the drug. However, from the pharmaceutical industry's perspective a personalised medicine approach entails a significant shift from their traditional blockbuster business model. In particular, a personalised medicine business model requires interdisciplinary collaboration between the traditionally separate diagnostics and pharmaceutical industries and recent attempts at such collaborations have had mixed results. For example, whilst DakoCytomation and Genentech successfully co-market HercepTest (a diagnostic which identifies patients who will benefit from Genentech's Herceptin for the treatment of HER2-positive metastatic breast cancer) a collaboration between Aventis and PharmaNetics to jointly develop the ENOX test for use with Aventis' drug enoxaprin has failed.

Thus, whilst personalised medicine provides significant medical advantages to patients, the logistic and commercial challenges of co-marketing of a diagnostic with a therapy mean that pharmaceutical companies must seriously consider whether a personalised medicine business model will provide sufficient commercial return to justify a change from their traditional blockbuster model. However, in view of the relatively recent emergence of personalised medicine, there is a lack of precedents for assessing such models. This makes it difficult for decision makers to estimate the return on investment (ROI) from a personalised medicine business plan. In particular, whilst there is a growing number of estimates and formulas addressing the impact of diagnostics on pharmaceutical R&D costs, there has not been a published assessment of the impact of diagnostics on future drug revenues.

### SUMMARY OF THE INVENTION

According to the invention there is provided a method of developing a personalised medicine business plan comprising the steps of:
obtaining a plurality of variables associated with an intended personalised medicine business plan;
generating a predicted revenue from the plurality of variables associated with the intended personalised medicine business plan;
collating the plurality of variables associated with the intended personalised medicine business plan and the predicted revenue to generate a hypothetical business scenario;
creating an archive of scenarios comprising data relating to personalised medicine, said data being acquired from real-world case studies;
comparing each of the scenarios in the archive with the hypothetical business scenario to identify a first archived scenario that most closely matches at least some of the variables of the hypothetical business scenario; and
extracting information from the first archived scenario, said information being used to justify the intended personalised medicine business plan and provide one or more guidance parameters on how to implement the intended personalised medicine business plan and thereby develop the intended personalised medicine business plan.

Preferably, the step of obtaining the plurality of variables associated with the intended personalised medicine business plan comprises a step of obtaining at least two of:
a percentage of patients taking a therapy that are most likely to benefit therefrom;
a percentage of patients taking a therapy that are likely to receive no benefit or be at risk of harm therefrom;
a diagnostic efficiency of a diagnostic test;
a price of the therapy;
a competitive market share advantage of the therapy;
a rate of patient adherence with the therapy;
a speed at which the therapy achieves a maximum market share;
and an effect of the diagnostic test on a launch date of the therapy.

Preferably, the step of generating the predicted revenue from the plurality of variables associated with the intended personalised medicine business plan comprises a step of calculating one or more sales of a combined therapy and theranostic.

Desirably, the step of calculating the sales of the combined therapy and theranostic comprises the steps of:
calculating a number of patients placed on the therapy; and calculating a potential market size for the therapy from an indication of the number of patients placed on the therapy and an allocated value for a patient.

Desirably, the step of generating the predicted revenue from the plurality of variables associated with the intended personalised medicine business plan comprises the additional steps of:
calculating an uptake of the theranostic; and
correcting the sales of the combined therapy and theranostic in accordance with the uptake of the theranostic.

Preferably, the step of calculating the uptake of the theranostic comprises the steps of:
calculating an opportunity to test index from the plurality of variables associated with the intended personalised medicine business plan; and
calculating a theranostic uptake increase slope from the opportunity to test index.

According to a second aspect of the invention there is provided a method of developing a personalised medicine business plan comprising the steps of:
obtaining a plurality of variables associated with an intended personalised medicine business plan;
obtaining a desired revenue from the intended personalised medicine business plan;
adjusting at least some of the plurality of variables associated with the intended personalised medicine business plan to achieve the desired revenue and to generate a plurality of adjusted variables associated with the intended personalised medicine business plan;
collating the plurality of adjusted variables associated with the intended personalised medicine business plan and the desired revenue to generate a hypothetical business scenario;
creating an archive of scenarios comprising data relating to personalised medicine, said data being acquired from real-world case studies;
comparing each of the scenarios in the archive with the hypothetical business scenario to identify a first archived scenario that most closely matches the hypothetical business scenario; and
extracting information from the first archived scenario, said information being used to justify the intended personalised medicine business plan and provide one or more guidance parameters on how to implement the intended personalised medicine business plan and thereby develop the intended personalised medicine business plan.

According to a third aspect of the invention there is provided a method of developing a personalised medicine business plan comprising the steps of:
obtaining a plurality of variables associated with an intended personalised medicine business plan;
obtaining a desired revenue from the intended personalised medicine business plan;
generating a predicted timescale within which the desired revenue will be achieved;
collating the plurality of variables associated with the intended personalised medicine business plan, the desired revenue and the predicted timescale to generate a hypothetical business scenario;
creating an archive of scenarios comprising data relating to personalised medicine, said data being acquired from real-world case studies;
comparing each of the scenarios in the archive with the hypothetical business scenario to identify a first archived scenario that most closely matches the hypothetical business scenario; and
extracting information from the first archived scenario, said information being used to justify the intended personalised medicine business plan and provide one or more guidance parameters on how to implement the intended personalised medicine business plan and thereby develop the intended personalised medicine business plan.

According to a fourth aspect of the invention there is provided a system for developing a personalised medicine business plan comprising:
a means of obtaining a obtaining a plurality of variables associated with an intended personalised medicine business plan;
a means of generating a predicted revenue from the plurality of variables associated with the intended personalised medicine business plan;
a means of collating the plurality of variables associated with the intended personalised medicine business plan and the predicted revenue to generate a hypothetical business scenario;
a means of creating an archive of scenarios comprising data relating to personalised medicine, said data being acquired from real-world case studies;
a means of comparing each of the scenarios in the archive with the hypothetical business scenario to identify a first archived scenario that most closely matches the hypothetical business scenario; and
a means of extracting information from the first archived scenario, said information being used to justify the intended personalised medicine business plan and provide a one or more guidance parameters on how to implement the intended personalised medicine business plan and thereby develop the intended personalised medicine business plan.

According to a fifth aspect of the invention there is provided a system for developing a personalised medicine business plan comprising:
a means of obtaining a plurality of variables associated with an intended personalised medicine business plan;
a means of obtaining a desired revenue from the intended personalised medicine business plan;
a means of adjusting at least some of the plurality of variables associated with the intended personalised medicine business plan to achieve the desired revenue and generate a plurality of adjusted variables associated with the intended personalised medicine business plan;
a means of collating the plurality of adjusted variables associated with the intended personalised medicine business plan and the predicted revenue to generate a hypothetical business scenario;
a means of creating an archive of scenarios comprising data relating to personalised medicine, said data being acquired from real-world case studies;
a means of comparing each of the scenarios in the archive with the hypothetical business scenario to identify a first archived scenario that most closely matches the hypothetical business scenario; and
a means of extracting information from the first archived scenario, said information being used to justify the intended personalised medicine business plan and provide a one or more guidance parameters on how to implement the intended personalised medicine business plan and thereby develop the intended personalised medicine business plan.

According to a sixth aspect of the invention there is provided a system for developing a personalised medicine business plan comprising:
a means of obtaining a plurality of variables associated with an intended personalised medicine business plan;
a means of obtaining a desired revenue from the intended personalised medicine business plan;
a means of generating a predicted timescale within which the desired revenue will be achieved;
a means of collating the plurality of variables associated with the intended personalised medicine business plan, the desired revenue and the predicted timescale to generate a hypothetical business scenario;
a means of creating an archive of scenarios comprising data relating to personalised medicine, said data being acquired from real-world case studies;
a means of comparing each of the scenarios in the archive with the hypothetical business scenario to identify a first archived scenario that most closely matches the hypothetical business scenario; and
a means of extracting information from the first archived scenario, said information being used to justify the intended personalised medicine business plan and provide a one or more guidance parameters on how to implement the intended personalised medicine business plan and thereby develop the intended personalised medicine business plan.

According to a seventh aspect of the invention there is provided a database comprising data formatted in a manner that complies with the fourth, or fifth or sixth aspects.

### BRIEF DESCRIPTION OF THE DRAWINGS

An embodiment of the invention is herein described, by way of example only, with reference to the accompanying Figures and Tables in which:

FIG. 1 is a block diagram providing an overview of the architecture of the method and system for developing a personalised medicine business plan in accordance with the present invention;

FIG. 2 is a block diagram of a predictive model and investment return model used in the method and system for developing a personalised medicine business plan shown in FIG. 1;

FIG. 3 is a block diagram providing a more detailed view of the calculation of combined sales of the desired therapy and theranostic in the method and system for developing a personalised medicine business plan shown in FIG. 1;

FIG. 4 is a block diagram providing a more detailed view of the calculation of the uptake of a theranostic in the method and system for developing a personalised medicine business plan shown in FIG. 1;

FIG. 5 is a block diagram providing a more detailed view of the calculations performed in an investment return model in the uptake of a theranostic in the method and system for developing a personalised medicine business plan shown in FIG. 1;

FIG. 6 is a bar chart comparing a prediction of the number of patients treated with a therapy made by a traditional pharmaceutical business model and the method and system for developing a personalised medicine business plan of the present invention;

Table 1 shows base case and test-adjusted case settings for personalised medicine revenue drivers in a hypothetical example relating to the diagnosis and treatment of genital herpes;

Table 2 shows a series of test uptake values used for developing a series of test uptake scenarios to test the method and system for developing a personalised medicine business plan of the present invention on a hypothetical example relating to the diagnosis and treatment of genital herpes; and

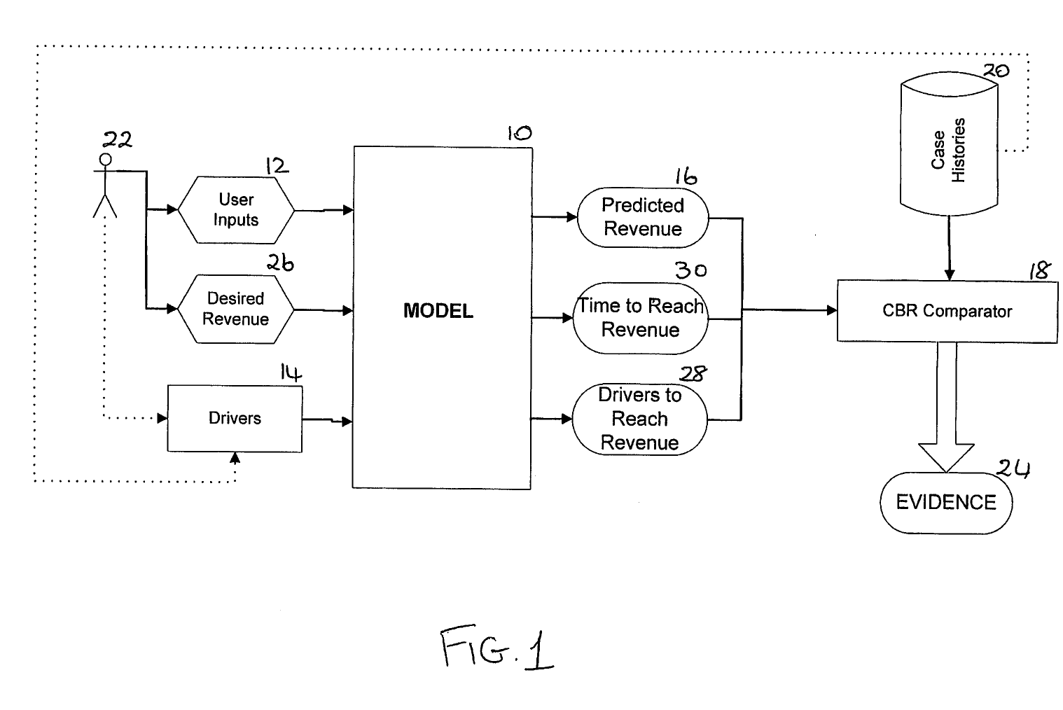

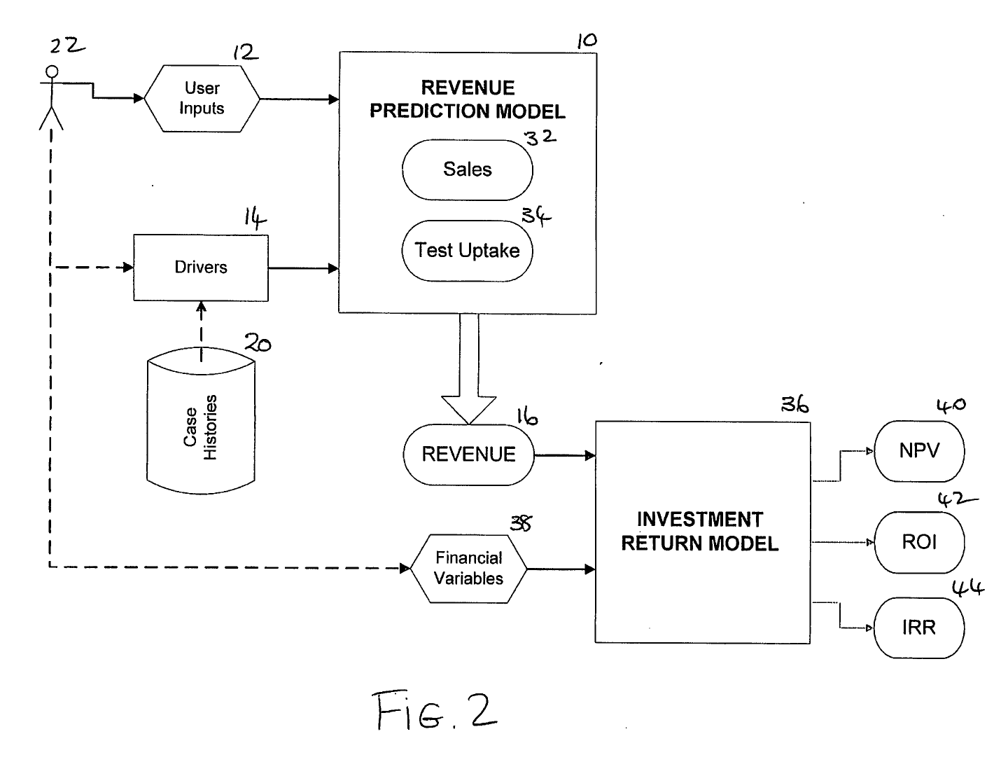

Tables 3a, 3b and 3c comprises profit and loss sheets generated by the method of the present invention using the personalised medicine revenue drivers (shown in FIG. 1) with each of the three uptake scenarios shown in Table 2.

For simplicity, in FIGS. 3 to 5, user inputs to the method and system for developing a personalised medicine business plan are depicted as shaded hexagonals, personalised medicine revenue drivers are shown as clear boxes and variables calculated in the method are shown as shaded ovals.

### DETAILED DESCRIPTION OF THE PREFERRED EMBODIMENT

The following discussion will provide a broad overview of the architecture and function of the present invention. This will be followed by a brief description of key variables (or personalised medicine revenue drivers) used in the present invention. The discussion will continue with a more detailed discussion of the calculations performed during a first and second operational phase of the invention. The discussion will finish with a description some experimental results generated by the method of the present invention.

### 1. Overview

The present invention uses references from the literature on medicine, pharmaceutical marketing and diffusion theory applied to the healthcare industry to develop a hybrid predictive-modelling case-based reasoning approach for predicting revenue from a personalized medicine business plan and validating the same. More particularly, and referring to FIG. 1, the present invention comprises a revenue prediction model 10 (which represents the relationship between a plurality of pharmaceutical and diagnostic business variables 12, 14 and the revenue 16 generated therefrom) and a case-based reasoning (CBR) comparator 18 (for validating an intended personalized medicine business plan with reference to precedents stored in a case-study archive 20).

The present invention has two operational modes. In the first operational mode, the revenue prediction model 10 is provided (by a user 22) with values 12 from an intended personalized medicine business plan. The revenue prediction model 10 is also provided with values of variables (known as personalized medicine revenue drivers 14) that have been previously identified as being key to determining the revenue from the joint marketing of a therapy and theranostic. The values of the personalized medicine revenue drivers 14 may be provided by the user 22 or may be calculated from the precedents in the case-study archive 20. The revenue prediction model 10 integrates the personalized medicine revenue drivers 14 and user-inputted variables 12 in a tailored, non-linear fashion to predict the revenue 16 from the intended personalized medicine business plan. The complex non-linear relationships represented in the revenue prediction model 10 contrast with the simpler linear (cascade) relationship models traditionally used in pharmaceutical modelling and prevents the present invention from over-simplifying the impact of a theranostic on a therapy. The revenue 16 is forwarded to an investment return model (not shown) whose function will be described later.

The CBR comparator 18 collates the user-inputted values 12, personalized medicine revenue drivers 14 and predicted revenue 16 into a hypothetical business scenario. The CBR comparator 18 compares the hypothetical business scenario with the precedents stored in the case-study archive 20 to identify the precedent that most closely matches the hypothetical business scenario. Details are extracted from the closest matching precedent to provide supporting evidence 24 for the hypothetical business scenario and guidance on how the intended personalized medicine business plan might be achieved. More particularly, chosen values for the variables used to generate the business scenario are compared with values from the case-study archive 20 to justify/validate the values chose for said variables. This contrasts with traditional pharmaceutical business models that focus on revenue prediction and do not provide any guidance on strategies for achieving the required financial target.

In the second operational mode, the revenue prediction mode 10 is provided with user inputted values 12 from an intended personalized medicine business plan and the revenue 26 desired therefrom. The revenue prediction model 10 uses these variables to calculate the values of the personalized medicine revenue drivers 28 required to reach the desired revenue 26 and/or the timescale 30 in which the desired revenue 26 can be achieved.

As in the first operational mode, the CBR comparator 18 collates the user-inputted values 12, calculated personalized medicine revenue drivers 28, desired revenue 26 and timescale 30 into a hypothetical business scenario. The CBR comparator 18 identifies a precedent from the case-study archive 20 that most closely matches the hypothetical business scenario and extracts details therefrom to provide evidence 24 in support of the hypothetical business scenario (and guidance on how the intended personalized medicine business plan might be achieved).

The personalised medicine revenue drivers 14, 28 of the present invention are derived from a thorough understanding of both the diagnostics and pharmaceutical markets and contrast with the drivers used in traditional pharmaceutical modelling that do not consider a test's impact on therapy revenue. Furthermore, since the revenue prediction model 10 includes multiple drivers of theranostic impact on therapy, the model embraces the highly variable and unpredictable nature of diagnostic clinical utilization as it impacts a linked therapy. This contrasts with traditional pharmaceutical revenue models, which assume that inefficiencies in the diagnostic market are not a major factor in determining the revenue from a therapy.

The revenue prediction model 10 is developed using “real-world” or historical, peer-reviewed, published case-study data. Similarly, the CBR comparator 18 validates predictions from the prediction model 10 using precedents (e.g. from a seven year revenue stream for a blockbuster drug and a nine-year revenue stream for a speciality therapy) in the case-study archive 20. These two features further differentiate the present invention from traditional pharmaceutical revenue prediction models that are based on market research data (of hypothetical future doctor reaction to drug value propositions) and tend to be highly subjective (and/or fail to reflect real market conditions). More particularly, by being based on actual “real-world” historical data, the present invention removes the uncertainty and subjectivity associated with traditional pharmaceutical revenue prediction models and provides the pharmaceutical industry with a peer-reviewed, validated, repeatable, benchmarked methodology for the repeated assessment of diagnostic impact on a targeted therapy, which more realistically reflects the market conditions of personalized medicine.

Whilst one of the primary functions of the present invention is to predict the revenue obtainable by allying a therapy with a theranostic, the invention also generates estimates of other financial variables which describe the overall shape of the market impact of the therapy-theranostic alliance and are instrumental in understanding how a predicted revenue may be achieved. The other such financial variables include estimates of:
the impact on a diagnostic partner of a proposed co-marketing strategy;
the incentives required to drive the diagnostic partner;
the impact of the proposed co-marketing strategy on the market size of a relevant diagnostic;
the impact of the proposed co-marketing strategy on the competitive dynamics of the relevant therapy market;
the cost of marketing the diagnostic to ensure therapy sales are met;
the size of the sales force required to promote the diagnostic; and
the numbers of doctors required to drive use of the diagnostic.

For example, consideration of different scenarios might determine what factor affecting sales of the therapy the theranostic is chosen to impact (e.g. diagnostic efficiency, adherence).

The modeling methodology of the present invention enables further case studies to be added to the case-study archive 20 as they become available. This in turn will lead to an improvement in the performance of the CBR comparator 18 and the provision of more accurate business intelligence to users. Accordingly, the present invention contrasts markedly with traditional pharmaceutical modelling methodologies, in which one-off models are built and the knowledge acquired therein is not carried over to other models. Furthermore, since the modelling methodology of the present invention is not tied to a specific medical disorder, the present invention can be generalised to a wide variety of therapeutic areas.

### 2. Personalised Medicine Revenue Drivers

The variables used as personalised medicine revenue drivers are described in more detail below. However, it will be appreciated that the present invention is not limited to these specific variables and could instead be implemented with any other suitable variables. In particular, additional personalised medicine revenue drivers may include the erosion of sales of a given therapy and/or theranostic after Patent protection therefor has expired.

2(a) Responders (Percentage of Patients Taking a Test Who are Most Likely to Benefit from the Therapy)

The ability to identify patients most likely to benefit from a drug prior to initiating therapy can have both positive and negative effects on a drug's profitability. In particular, the ability to identify patients more likely to respond to a therapy prior to enrolment can significantly reduce the size, duration and cost of a clinical trial. On the other hand, such pre-screening reduces the potential patient population for a drug, particularly if third-party payers begin to mandate screening in an effort to control high drug costs.

2(b) Screening Effect (Diagnostic Efficiency)

The ability to properly diagnose patients and begin therapy is a critical variable in the market size of a drug. Consequently, a diagnostic, that is targeted toward a normally under-diagnosed disease can have a positive effect on the overall market size of the corresponding therapy. In the present invention, diagnostic efficiency is determined through an estimate of the number of patients diagnosed with a condition using a given test or test modality compared to an estimate of the total number of patients both diagnosed and undiagnosed with that condition (using other inferior testing methodologies or empirical analysis).

2(c) Price Premium (Effect of a Diagnostic on the Pricing of a Therapy)

Despite the reduction in market size produced by targeting therapies, it may be possible to demonstrate significant therapeutic advantage to such targeted groups, wherein this advantage is sufficient to support premium pricing for the relevant therapy.

2(d) Competitive Market Share Advantage (Percentage Share of Total Available Market)

Targeted therapies provide companies with an opportunity to gain an advantage in the competition for market share that is not tied to promotional strategies. In particular, targeted therapies offer physicians a clinical basis from which to determine the best treatment option for their patients. This in turn, increases switching costs for the patient and physician and reduces the likelihood of losing market share to similar drugs. Similarly, in smaller therapeutic areas, or areas that feature less competition, a targeted therapy offers the potential of gaining market share from drugs that arrived earlier to the market and would otherwise hold the greatest share thereof. In the present invention, competitive advantage data is taken from published data for over 129 pharmaceutical cases (Grabowski and Vernon) and modulated according to whether the therapy achieves top decile, second decile or average market share.

2(e) Rate of Patient Adherence with Therapy and Effect of Intervention Thereon

A significant factor in patient adherence is the perception of benefit from a therapy. Many patients stop taking their therapy because they do not perceive the lack of a negative effect as a significant positive benefit. Clinical evidence from diabetes, HIV and coagulation management studies suggests that when diagnostic monitoring tools make patients aware of their progress, their adherence improves. Thus, it can be beneficial for a pharmaceutical company to seek a closer alignment with diagnostic monitoring tools, either to help achieve correct therapeutic dosages or to make patients feel that they are in control of their condition or improving, thereby encouraging patient adherence and driving revenue.

2(f) Early Adoption (Speed at which a Therapy Achieves its Maximum Market Share)

A novel therapy can take three to four years (or more) to be adopted. Thus, this process has a direct impact on when a drug will reach its peak-year sales. The availability of a diagnostic that identifies a patient's candidacy for therapy (or demonstrates the value of treatment through post-therapy monitoring) removes some of the uncertainty associated with a novel therapy, thereby influencing adoption rate and revenue of the new therapy. In other words, with a theranostic approach, adopters who would otherwise have waited for evidence that a drug would work for a particular patient will adopt the drug earlier, because the patients for whom it has been proven that the therapy will work have already been identified by the test (thereby removing the need to “wait and see”).

2(g) Early Launch (Effect of Diagnostic on Launch Date of Therapy)

Diagnostics could cut the time from target identification to drug launch from 10-12 years to 3-5 years, thus reducing pre-launch development costs per drug to about $200 million. This is achieved because the availability of a test will reduce the number of patients required to power a clinical trial, thereby accelerating the trial, facilitating regulatory approval and thus reducing the overall time to market.

### 3. Detailed Description of the Calculations Performed During the First Operational Phase of the Present Invention

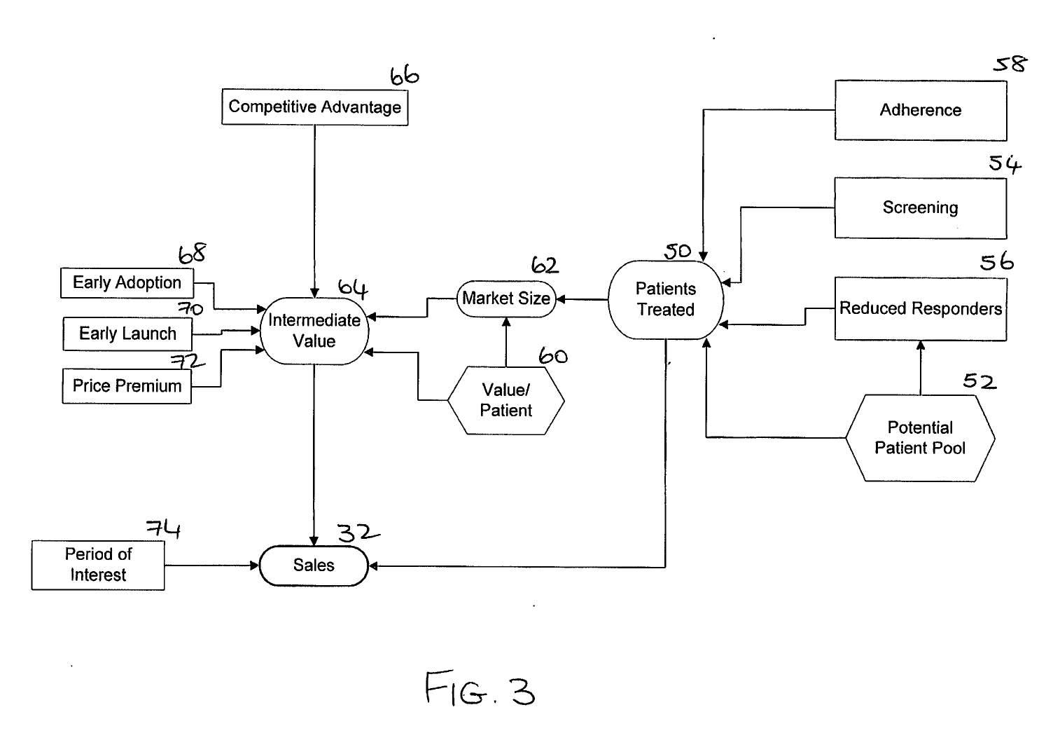

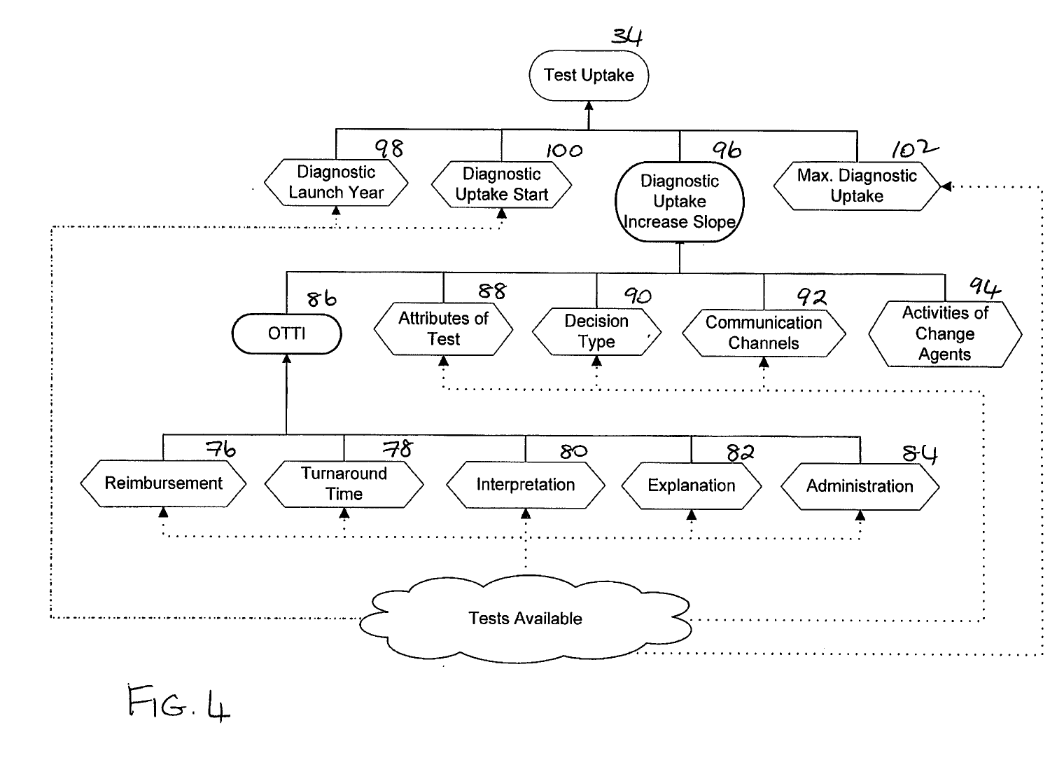

Referring to FIG. 2, during the first operational phase of the present invention, the values of personalised medicine revenue drivers 14 may be obtained from the minimum, maximum and mean values of relevant variables in the precedents stored in the case-study archive 20. Alternatively, values for the personalised medicine revenue drivers 14 may be provided by the user 22, in which case, a warning is issued to the user 22 if they select a value outside the range supported by the precedents (in the case-study archive 20). Similarly, a warning may be issued if a particular value or combination of values provided by the user causes a logic error (e.g. adherence >1).

The personalised medicine revenue drivers 14 and other user inputted values 12 are used in the revenue prediction model 10 to calculate the combined sales 32 of the desired therapy and theranostic. The revenue prediction model 10 also calculates the uptake 34 of the theranostic and corrects the combined sales 32 calculation accordingly. The corrected sales figures are used to calculate the revenue from the joint marketing of the therapy and theranostic.

The predicted revenue 16 is then forwarded to an investment return model 36, whereupon it is combined with other financial variables 38 provided by the user to calculate the net present value (NPV) 40, return on investment (ROI) 42 and internal rate of return (IRR) 44 from the intended personalised medicine business plan. The NPV 40, ROI 42 and IRR 44 variables are output to the user in graphical or tabular format of value per year from launch; or as a series of scenarios calculated over a given time period. It will be appreciated that the present invention is not limited to these NPV, ROI and IRR output variables and could instead provide other metrics for assessing investment benefits.

### 3.1 Calculation of Combined Sales of Therapy and Theranostic

A more detailed analysis of the operations leading to the calculation of the combined sales of the desired therapy and theranostic is shown in FIG. 3. In particular, the number of patients 50 who might be placed on a desired therapy is calculated from the user input of the number of patients with the corresponding condition 52 and the personalised medicine revenue drivers comprising the screening effect 54, reduced responders 56, and adherence to therapy rate 58. The number of patients 50 who might be placed on the desired therapy is then combined with the user input of the value of a patient 60 to calculate the potential market size 62 for the therapy.

An intermediate value 64 is calculated from the potential market size 62 and the personalised medicine revenue drivers comprising the competitive advantage 66, early adoption 68, effect of the theranostic on the launch date 70 of the therapy and the effect of the theranostic on the pricing 72 of the therapy 72. The intermediate value 64 is in turn used to calculate the sales 32 of the desired therapy and theranostic. The sales may be calculated for a user selected period of time 74 encompassing the entire sales cycle of the therapy or a portion thereof.

### 3.2 Calculation of Theranostic Uptake

A more detailed analysis of the operations leading to the calculation of the theranostic uptake 34 is shown in FIG. 4. In particular, user inputs of reimbursement 76, turnaround time 78, requirement for interpretation of results 80, requirement for extensive patient interaction 82 and integration with provider's administration 84 and compliance activities, are used to calculate an opportunity to test index (OTTI) 86. The OTTI 86 is combined with user inputs of the attributes of the test 88, decision type 90, communication channels 92 and activities of change agents 94 to calculate a diagnostic uptake increase slope (%/year) 96. The calculated diagnostic uptake increase slope (%/year) 96 is combined with user inputs of diagnostic launch year 98, diagnostic uptake start value (%) 100, and maximum level of diagnostic uptake 102 to calculate year on year uptake 34 of the theranostic. As discussed, the theranostic uptake 34 is used to correct the combined sales of the theranostic and therapy for a less than 100% uptake of the theranostic.

### 3.3 Calculations Performed in the Investment Return Model

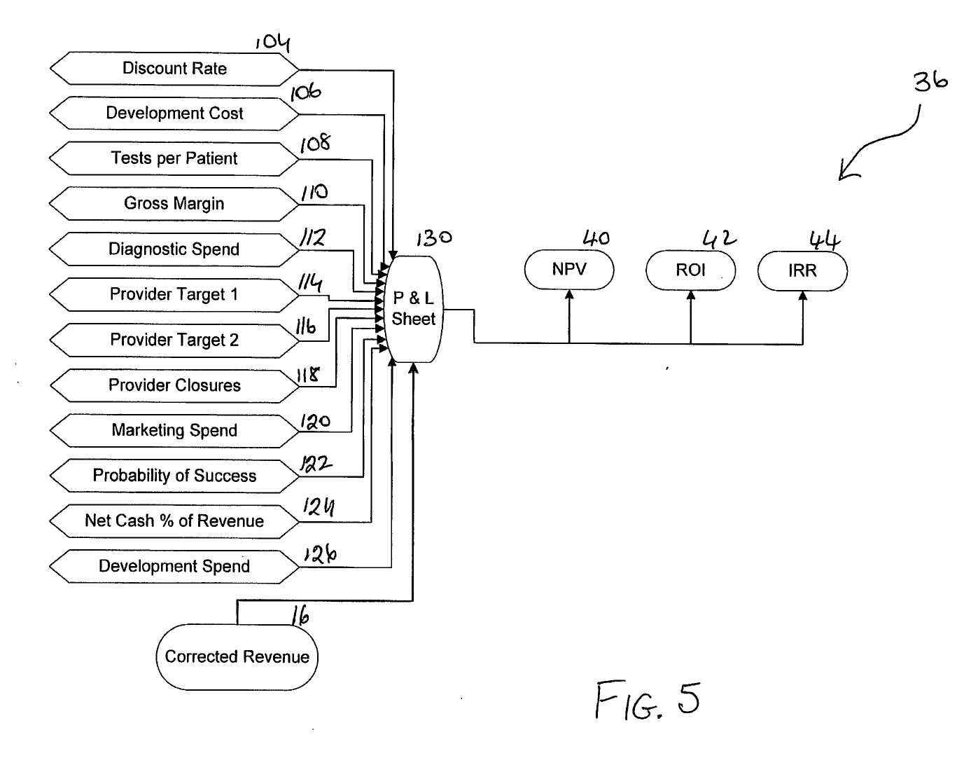

FIG. 5 provides a more detailed analysis of the calculations performed in the investment return model 36 of FIG. 2. In particular, user inputs of estimated discount rate 104, estimated development cost 106, tests per patient 108, gross margin (%) 110, diagnostic spend (% of therapeutic spend) 112, provider target 114, provider target 2 116, provider closures per sales representative per year 118, marketing spend 120 per year, probability of diagnostic success (%) 122, net cash % of revenue (%) 124, and theranostic development spend ($M) 126 are combined with the corrected revenue 16 from the combined sales of the theranostic and therapy (calculated from the revenue prediction model (not shown)) to construct profit and loss sheets 130. The profit and loss sheets 130 are used in turn to the calculate net present value (NPV) 100, return on investment (ROI) 102 and internal rate of return (IRR) 104 of the intended personalised medicine business plan. As an aside, the marketing spend per year 120 is calculated from estimates of the degree of provider conversion (i.e. converting a non-test using provider to a test using provider) and the marketing resources (in terms of representatives) required to achieve the selected degree of conversion.

### 4. Detailed Description of the Calculations Performed During the Second Operational Phase of the Present Invention

As previously mentioned, during the second operational mode of the present invention, the revenue prediction model is provided with user inputted values from an intended personalized medicine business plan and the revenue desired therefrom. The revenue prediction model uses these variables to calculate the values of the personalized medicine revenue drivers (within their case limits) required to reach the desired revenue (e.g. $300 m in year 3 sales). To achieve this, the values of the personalized medicine revenue drivers are seeded with mean values from the case-study archive and then adjusted through a feedback loop in accordance with the:
closeness of fit between a predicted and desired value for revenue, ROI, NPV and IRR;
effect of marketing or other activities to reduce a difference between predicted and desired values for revenue, ROI, NPV and IRR;
the sensitivity of revenue, ROI, NPV and IRR to variation in estimates of individual personalised medicine revenue drivers.

In addition, as previously discussed, during the second operational phase, the revenue prediction model may indicate that a target revenue is not achievable or not achievable within the required time interval (e.g. will take 5 years rather than the desired 3). Furthermore, the personalized medicine revenue drivers may be fixed to the mean values (from the case history archive) and the revenue prediction model used to show the revenue generated using such mean values. Alternatively, the personalized medicine revenue drivers may be fixed to maximum and minimum values (from the case history archive) and the revenue prediction model used to show the span of achievable revenues therewith.

### 5. Experimental Results

5.1 General Example

The method of the present invention was used to predict the number of patients treated with a particular therapy (wherein the prediction also considers the effect of the sales of a theranostic for the relevant condition). The prediction from the method of the present invention was compared with a similar prediction made by a traditional pharmaceutical forecast model (that does not take into account the effect of a theranostic).

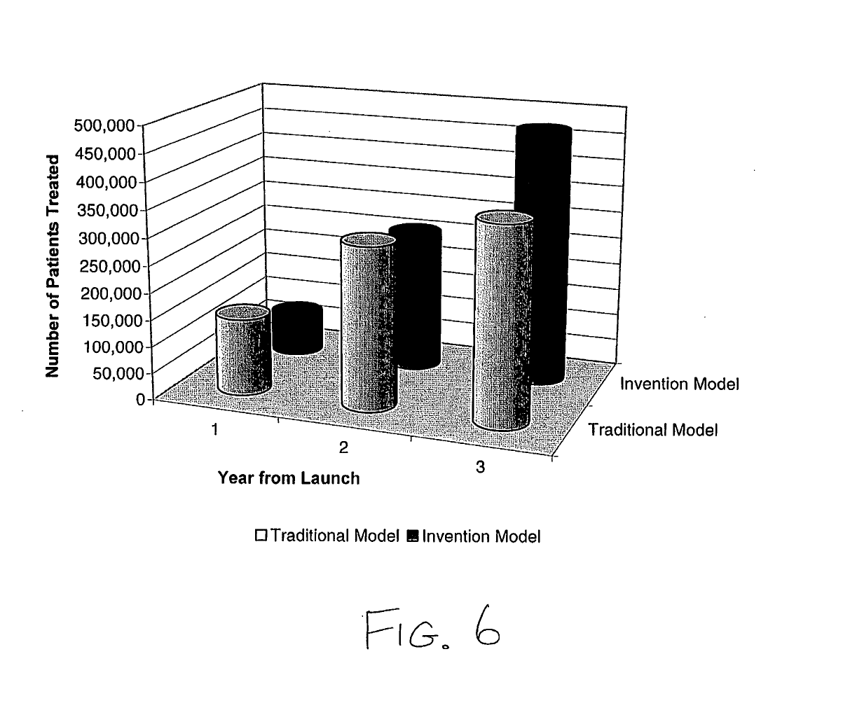

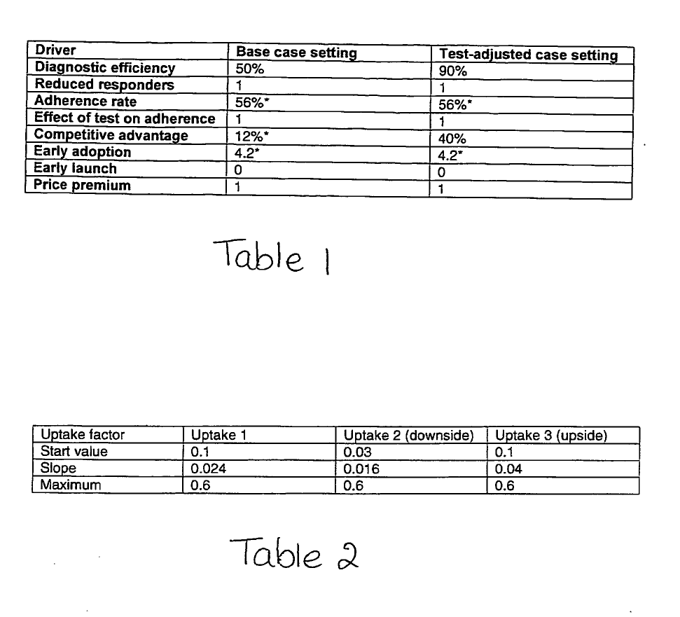

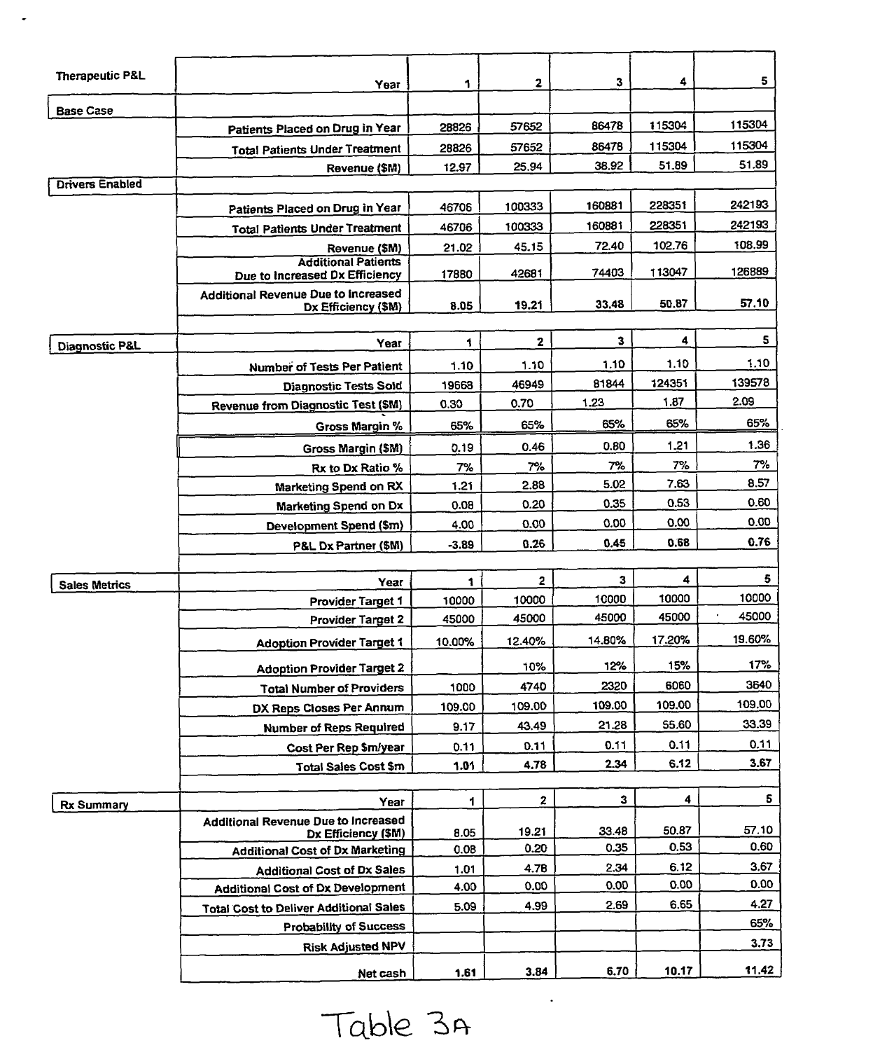

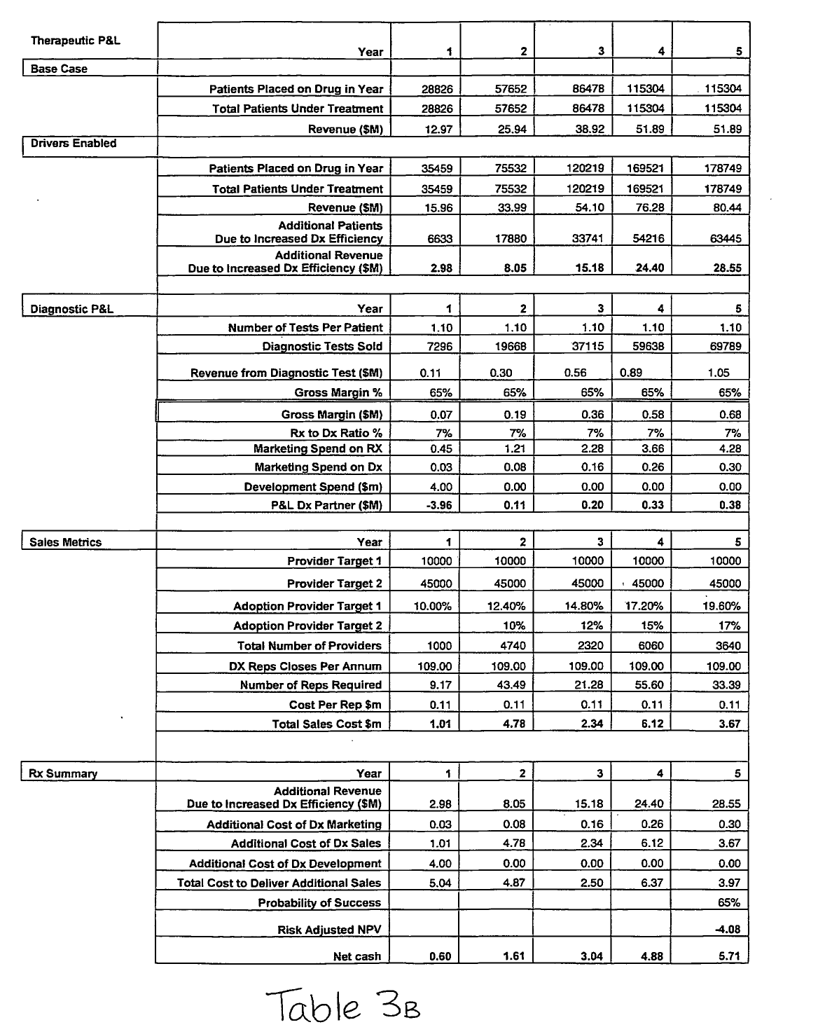

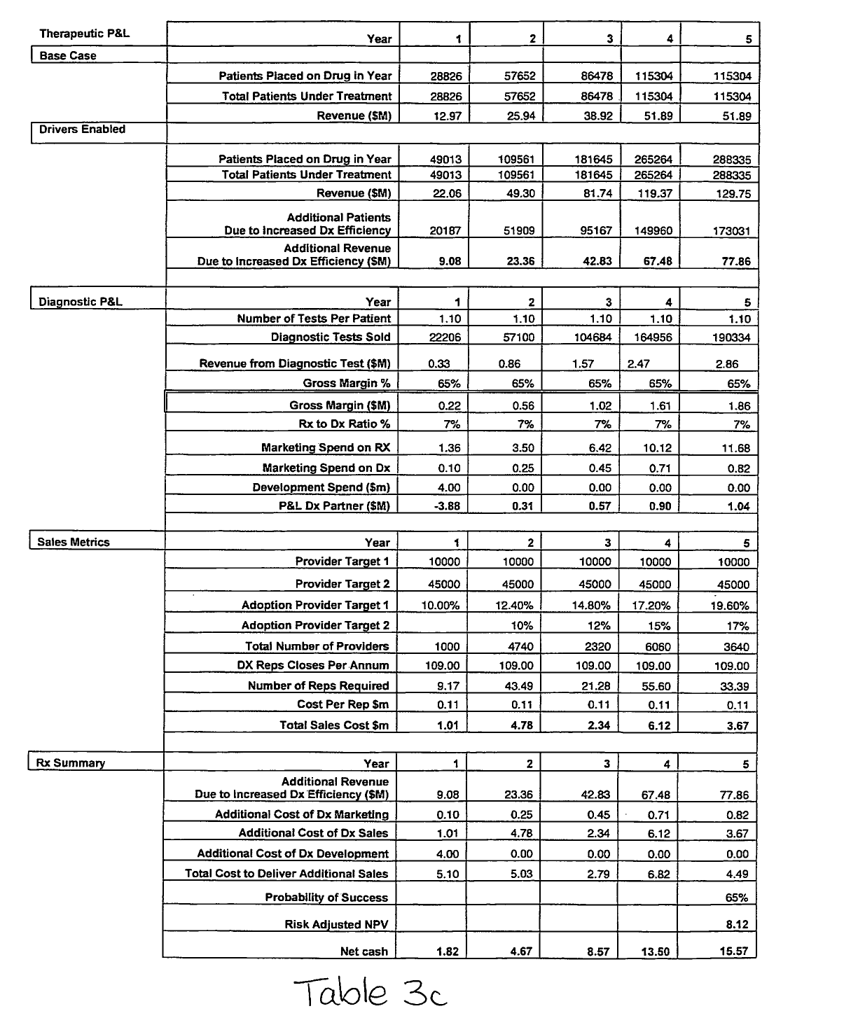

Referring to FIG. 6, the number of patients treated between the first and third years predicted by the method of the present invention is similar to that predicted by the traditional pharmaceutical forecast model. However, the rate of increase of sales between years 1 and 2 predicted by the method of the present invention is significantly lower than that predicted by the traditional pharmaceutical forecast model.

The business intelligence provided by the CBR comparator of the present invention suggests that in order to achieve the penetration targets shown in FIG. 6, a minimum of 35% of target providers will need to act as innovators and early adopters of the theranostic by the end of year 1, using the theranostic on 95% of their patients. This contrasts with the traditional pharmaceutical modelling approach which estimates that 69% of providers should use the theranostic 48% of the time.

More particularly, the business intelligence provided by the CBR comparator of the present invention suggests that in order to achieve the penetration targets shown in FIG. 6:
an estimated £7.6 m must be spent on marketing the theranostic at the pre-launch and year 1 stages;
an estimated direct sales force of 84 sales representatives in the US and 20, sales representatives in Europe is required to accelerate adoption of the theranostic through education;
an estimated minimum of 6 laboratories geographically spread in the US and 1 per major European city, must offer the test with a turnaround time of no less than 4 days; and
the test must cost no more than $100.

The method of the present invention was also used to optimise resource requirements as opposed to test uptake scenarios. The results from this exercise suggested that the number of patients treated between 2010 and 2012 could be increased by 11%. However, the increase in sales in years 1 and 2 predicted by the method of the present invention remains lower than that predicted by a traditional pharmaceutical forecast model.

The business intelligence provided by the CBR comparator of the present invention suggests that in order to achieve the desired penetration targets, a minimum of 40% of target providers must behave as innovators and early adopters of the theranostic by the end of year 1, using this on 95% of their patients. More particularly, the business intelligence provided by the CBR comparator of the present invention suggests that in order to deliver this best in class market penetration:
an estimated £8.3 m must be spent in marketing the theranostic at the prelaunch and year 1 stages;
an estimated direct sales force of 94 sales representatives in the US and 30 in Europe is required to accelerate adoption of the theranostic through education; and
a “validation of process” trial and peer-to-peer communication program must be implemented at the pre-launch stage to familiarise providers with the theranostic.

The above modelling exercise has confirmed the benefit to a therapy of focusing resources on sales of the theranostic as early as possible. In particular, the modelling exercise showed that approximately every 1$ spent on theranostic sales and marketing to innovators and early adopter providers, translates into $5 in therapy sales. This compares well with successful direct to customer advertising campaigns, wherein $3 revenue is generated for every $1 spent on TV advertising.

The method of the present invention was also used to predict the benefit to a laboratory partner of supporting a therapy. The method of the present invention estimated that the testing market value will increase significantly, offering between £34-£50 m contribution to overheads (assuming 65% profit margins) to laboratories servicing this market.

5.2 Genital Herpes Example

Genital herpes is estimated to affect, on average, 25% of the US adult population (i.e. approx. 50 million people). The vast majority (approximately 90%) of these people do not know they are infected. Thus, there are approximately 45 million US adults who have genital herpes, but are unaware of it. Of these people, approximately 25% will be truly asymptomatic. Thus, approximately 34 million US adults will display some form of symptoms at some time during a year. Assuming people will only present to their physician during an outbreak of the disease, and assuming 5 outbreaks of 7 days duration per year on average; approximately 3.4 million people per year will present to their physician for testing (however, in reality, the figure is considerably higher).

The standard diagnostic workup for genital herpes involves clinical examination, taking a patient history and viral culture of lesions (if present). The standard diagnostic approach has a relatively poor sensitivity of around 50%. However, a new means of diagnosing genital herpes caused by HSV-2 (around 90% of cases) has been recently developed using type specific serology (TSS), wherein TSS has a much higher sensitivity (typically >90%).

At present, there are no drugs available to cure herpes. However, for the sake of example, assume a company X produces a novel drug, Simplavir, that eradicates the virus from a patient. In this hypothetical example, a one year course of Simplavir (necessary to give >90% clinical efficacy) produces $450 per patient for the company X. The present invention is used to advise company X on how shifting the diagnostic paradigm away from exam and culture to TSS could impact their sales of Simplavir.

The TSS test chosen is called Oran2; it is a lateral flow test designed to be used at the point of care (thereby eliminating the risk of patients not returning for the results of their test). Average sensitivity across all patients is 90%.

Table 1 shows base case and test-adjusted case settings for the personalised medicine revenue drivers (wherein the asterisk superscripts above values indicate that the value shown is a mean values from all the cases). Values for diagnostic efficiency in a base case and test-adjusted case are supported by 4 and 0 cases, respectively (the value for the test-adjusted diagnostic efficiency is flagged as beyond the range of values that the stored case-studies supports). A series of test uptake scenarios are then developed based on the values shown in Table 2.

The method of the present invention is then used to calculate profit and loss sheets for each of the three uptake scenarios (shown in Table 2), wherein the resulting profit and loss sheets are shown in Table 3.

Use of the most likely uptake scenario (novel marker, established platform: start value=0.1, slope=0.024, ceiling=0.6) suggests use of the test could generate an additional $168 M revenue over 5 years. Analysis of variation of NPV with slope (assuming a constant start value and ceiling of 0.1 and 0.6, respectively) indicates an uptake slope of 0.01 will be required in order for a positive NPV to be achieved.

Software, web, and computer readable data storage implementations of the various embodiments and method steps described herein can be accomplished with methods known in the art including programming techniques with rule based logic and other logic.

Alternatives and modifications may be made to the above without departing from the scope of the invention.

1. A method of developing a personalised medicine business plan comprising the steps of:
obtaining a plurality of variables associated with an intended personalised medicine business plan;
generating a predicted revenue from the plurality of variables associated with the intended personalised medicine business plan;
collating the plurality of variables associated with the intended personalised medicine business plan and the predicted revenue to generate a hypothetical business scenario;
creating an archive of scenarios comprising data relating to personalised medicine, said data being acquired from real-world case studies;
comparing each of the scenarios in the archive with the hypothetical business scenario to identify a first archived scenario that most closely matches at least some of the variables of the hypothetical business scenario; and
extracting information from the first archived scenario, said information being used to justify the intended personalised medicine business plan and provide one or more guidance parameters on how to implement the intended personalised medicine business plan and thereby develop the intended personalised medicine business plan.

2. The method of claim 1, wherein the step of obtaining the plurality of variables associated with the intended personalised medicine business plan comprises a step of obtaining at least two of:
a percentage of patients taking a therapy that are most likely to benefit therefrom;
a percentage of patients taking a therapy that are likely to receive no benefit or be at risk of harm therefrom;
a diagnostic efficiency of a diagnostic test;
a price of the therapy;
a competitive market share advantage of the therapy;
a rate of patient adherence with the therapy;
a speed at which the therapy achieves a maximum market share;
and an effect of the diagnostic test on a launch date of the therapy.

3. The method of claim 1, wherein the step of obtaining the plurality of variables associated with the intended personalised medicine business plan comprises a step of obtaining an erosion of one or more sales of the therapy after a Patent therefor has expired.

4. The method of claim 1, wherein the step of obtaining the plurality of variables associated with the intended personalised medicine business plan comprises a step of obtaining an erosion of one or more sales of the diagnostic test after a Patent therefor has expired.

5. The method of claim 1, wherein the step of obtaining the plurality of variables associated with the intended personalised medicine business plan comprises a step of obtaining at least some of the plurality of variables associated with the intended personalised medicine business plan from a user.

6. The method of claim 5, wherein the method comprises an additional step of issuing an alarm in the event that a value of a one or more of the plurality of variables associated with the intended personalised medicine business plan exceeds a maximum value of one or more corresponding variables in the archive of scenarios.

7. The method of claim 5, wherein the method comprises an additional step of issuing an alarm in the event that a value of a one or more of the plurality of variables associated with the intended personalised medicine business plan is less than a minimum value of one or more corresponding variables in the archive of scenarios.

8. The method of claim 1, wherein the step of obtaining a plurality of variables associated with the intended personalised medicine business plan comprises a step of obtaining at least some of the plurality of variables associated with the intended personalised medicine business plan from the archive of scenarios.

9. The method of claim 8, wherein the step of obtaining at least some of the plurality of variables associated with the intended personalised medicine business plan from the archive of scenarios, comprises a step of calculating an average value of each of at least some of a plurality of variables comprised in the archive of scenarios.

10. The method of claim 8, wherein the step of obtaining at least some of the plurality of variables associated with the intended personalised medicine business plan from the archive of scenarios, comprises a step of calculating a maximum value of each of at least some of a plurality of variables comprised in the archive of scenarios.

11. The method of claim 8, wherein the step of obtaining at least some of the plurality of variables associated with the intended personalised medicine business plan from the archive of scenarios, comprises a step of calculating a minimum value of each of at least some of a plurality of variables comprised in the archive of scenarios.

12. The method of claim 1, wherein the step of obtaining a plurality of variables associated with the intended personalised medicine business plan, comprises a step of obtaining at least some of the plurality of variables associated with the intended personalised medicine business plan from the archive of scenarios and at least some of the plurality of variables associated with the intended personalised medicine business plan from a user.

13. The method of claim 1, wherein the step of generating the predicted revenue from the plurality of variables associated with the intended personalised medicine business plan comprises a step of calculating one or more sales of a combined therapy and theranostic.

14. The method of claim 13, wherein the step of calculating the sales of the combined therapy and theranostic comprises the steps of:
calculating a number of patients placed on the therapy; and
calculating a potential market size for the therapy from an indication of the number of patients placed on the therapy and an allocated value for a patient.

15. The method of claim 13, wherein the step of generating the predicted revenue from the plurality of variables associated with the intended personalised medicine business plan comprises the additional steps of:
calculating an uptake of the theranostic; and
correcting the sales of the combined therapy and theranostic in accordance with the uptake of the theranostic.

16. The method of claim 15, wherein the step of calculating the uptake of the theranostic comprises the steps of:
calculating an opportunity to test index from the plurality of variables associated with the intended personalised medicine business plan; and
calculating a theranostic uptake increase slope from the opportunity to test index.

17. The method of claim 1, wherein the step of generating the predicted revenue from the plurality of variables associated with the intended personalised medicine business plan comprises a step of calculating one or more sales of the combined therapy and a theranostic for a user-selected period of time encompassing at least some of a sales cycle of the therapy.

18. The method of claim 1, wherein the method comprises the additional steps of:
obtaining a plurality of financial variables from a user; and
generating a profit and loss sheet from the financial variables and the predicted revenue.

19. The method of claim 18, wherein the method comprises an additional step of calculating a return on investment from the profit and loss sheet.

20. The method of claim 19, comprising a step of presenting the return on investment in a form selected from the set comprising graphical form, tabular form and one or more scenarios calculated over a pre-defined time period.

21. The method of claim 18, wherein the method comprises an additional step of calculating a net present value from the profit and loss sheet.

22. The method of claim 18, wherein the method comprises an additional step of calculating an internal rate of return from the profit and loss sheet.

23. A method of developing a personalised medicine business plan comprising the steps of:
obtaining a plurality of variables associated with an intended personalised medicine business plan;
obtaining a desired revenue from the intended personalised medicine business plan;
adjusting at least some of the plurality of variables associated with the intended personalised medicine business plan to achieve the desired revenue and to generate a plurality of adjusted variables associated with the intended personalised medicine business plan;
collating the plurality of adjusted variables associated with the intended personalised medicine business plan and the desired revenue to generate a hypothetical business scenario;
creating an archive of scenarios comprising data relating to personalised medicine, said data being acquired from real-world case studies;
comparing each of the scenarios in the archive with the hypothetical business scenario to identify a first archived scenario that most closely matches the hypothetical business scenario; and
extracting information from the first archived scenario, said information being used to justify the intended personalised medicine business plan and provide one or more guidance parameters on how to implement the intended personalised medicine business plan and thereby develop the intended personalised medicine business plan.

24. A method of developing a personalised medicine business plan comprising the steps of:
obtaining a plurality of variables associated with an intended personalised medicine business plan;
obtaining a desired revenue from the intended personalised medicine business plan;
generating a predicted timescale within which the desired revenue will be achieved;
collating the plurality of variables associated with the intended personalised medicine business plan, the desired revenue and the predicted timescale to generate a hypothetical business scenario;
creating an archive of scenarios comprising data relating to personalised medicine, said data being acquired from real-world case studies;
comparing each of the scenarios in the archive with the hypothetical business scenario to identify a first archived scenario that most closely matches the hypothetical business scenario; and
extracting information from the first archived scenario, said information being used to justify the intended personalised medicine business plan and provide one or more guidance parameters on how to implement the intended personalised medicine business plan and thereby develop the intended personalised medicine business plan.

25. A system for developing a personalised medicine business plan comprising:
a first module adapted to obtain a plurality of variables associated with an intended personalised medicine business plan;
a second module adapted to generate a predicted revenue from the plurality of variables associated with the intended personalised medicine business plan;
a third module adapted to collate the plurality of variables associated with the intended personalised medicine business plan and the predicted revenue to generate a hypothetical business scenario;
a fourth module adapted to create an archive of scenarios comprising data relating to personalised medicine, said data being acquired from real-world case studies;
a fifth module adapted to compare each of the scenarios in the archive with the hypothetical business scenario to identify a first archived scenario that most closely matches the hypothetical business scenario; and
a sixth module adapted to extract information from the first archived scenario, said information being used to justify the intended personalised medicine business plan and provide a one or more guidance parameters on how to implement the intended personalised medicine business plan and thereby develop the intended personalised medicine business plan.

26. The system of claim 25, wherein the plurality of variables associated with the intended personalised medicine business plan, comprises at least two of:
a percentage of patients taking a therapy that are most likely to benefit therefrom;
a percentage of patients taking a therapy that are likely to receive no benefit or be at risk of harm therefrom;
a diagnostic efficiency of a diagnostic test;
a price of the therapy;
a competitive market share advantage of the therapy;
a rate of patient adherence with the therapy;
a speed at which the therapy achieves its maximum market share;
and an effect of the diagnostic test on a launch date of the therapy.

27. The system of claim 25, wherein the plurality of variables associated with the intended personalised medicine business plan, comprises an erosion of one or more sales of the diagnostic test after a Patent therefor has expired.

28. The system of claim 25, wherein the plurality of variables associated with the intended personalised medicine business plan, comprises an erosion of one or more sales of the therapy after a Patent therefor has expired.

29. The system of claim 25, wherein at least some of the plurality of variables associated with the intended personalised medicine business plan are obtainable from a user.

30. The system of claim 29, wherein the system comprises an alarm module configured to issue an alarm in the event that a value of one or more of the variables associated with the intended personalised medicine business plan obtained from the user exceeds a maximum value of one or more corresponding variables in the archive of scenarios.

31. The system of claim 30, wherein the system comprises an alarm module configured to issue an alarm in the event that a value of one or more of the variables associated with the intended personalised medicine business plan obtained from the user is less than a minimum value of one or more corresponding variables in the archive of scenarios.

32. The system of claim 25, wherein at least some of the plurality of variables associated with the intended personalised medicine business plan are obtainable from the archive of scenarios.

33. The system of claim 25, wherein at least some of the plurality of variables associated with the intended personalised medicine business plan are obtainable from a calculation of an average value of each of at least some of a plurality of variables comprised in the archive of scenarios.

34. The system of claim 25, wherein at least some of the plurality of variables associated with the intended personalised medicine business plan are obtainable from a calculation of a maximum value of each of at least some of a plurality of variables comprised in the archive of scenarios.

35. The system of claim 25, wherein at least some of the plurality of variables associated with the intended personalised medicine business plan are obtainable from a calculation of a minimum value of each of at least some of a plurality of variables comprised in the archive of scenarios.

36. The system of claim 25, wherein at least some of the plurality of variables associated with the intended personalised medicine business plan are obtainable from the archive of scenarios and at least some of the plurality of variables associated with the intended personalised medicine business plan are obtainable from a user.

37. The system of claim 25, wherein the second module comprises a seventh module adapted to calculate one or more sales of a combined therapy and theranostic.

38. The system of claim 37, wherein the seventh module comprises:
an eighth module adapted to calculate a number of patients placed on the therapy; and
a ninth module adapted to calculate a potential market size for the therapy from the number of patients placed on the therapy and an allocated value for a patient

39. The system of claim 37, wherein the second module comprises:
a tenth module adapted to calculate an uptake of the theranostic;
and an eleventh module adapted to correct the sales of the combined therapy and theranostic in accordance with the uptake of the theranostic.

40. The system of claim 39, wherein the tenth module comprises:
a twelfth module adapted to calculate an opportunity to test index from the plurality of variables associated with the intended personalised medicine business plan; and
a thirteenth module adapted to calculate a theranostic uptake increase slope from the opportunity to test index.

41. The system of claim 25, wherein the system comprises:
a fourteenth module adapted to obtain a plurality of financial variables from a user; and
a fifteenth module adapted to generate a profit and loss sheet from the financial variables and the predicted revenue.

42. The system of claim 41, wherein the system comprises a sixteenth module adapted to calculate a return on investment from the profit and loss sheet.

43. The system of claim 41, wherein the system comprises a seventeenth module adapted to calculate a net present value from the profit and loss sheet.

44. The system of claim 41, wherein the system comprises an eighteenth module adapted to calculate an internal rate of return from the profit and loss sheet.

45. The system of claim 25, wherein the archive of scenarios is contained in a database within the system.

46. The system of claim 25, wherein the archive of scenarios is contained in a database provided externally to the system and the system comprises a means of accessing the database.

47. A system for developing a personalised medicine business plan comprising:
a first module adapted to obtain a plurality of variables associated with an intended personalised medicine business plan;
a second module adapted to obtain a desired revenue from the intended personalised medicine business plan;
a third module adapted to adjust at least some of the plurality of variables associated with the intended personalised medicine business plan to achieve the desired revenue and generate a plurality of adjusted variables associated with the intended personalised medicine business plan;
a fourth module adapted to collate the plurality of adjusted variables associated with the intended personalised medicine business plan and the predicted revenue to generate a hypothetical business scenario;
a fifth module adapted to create an archive of scenarios comprising data relating to personalised medicine, said data being acquired from real-world case studies;
a sixth module adapted to compare each of the scenarios in the archive with the hypothetical business scenario to identify a first archived scenario that most closely matches the hypothetical business scenario; and
a seventh module adapted to extract information from the first archived scenario, said information being used to justify the intended personalised medicine business plan and provide a one or more guidance parameters on how to implement the intended personalised medicine business plan and thereby develop the intended personalised medicine business plan.

48. A system for developing a personalised medicine business plan comprising:
a first module adapted to obtain a plurality of variables associated with an intended personalised medicine business plan;
a second module adapted to obtain a desired revenue from the intended personalised medicine business plan;
a third module adapted to generate a predicted timescale within which the desired revenue will be achieved;
a fourth module adapted to collate the plurality of variables associated with the intended personalised medicine business plan, the desired revenue and the predicted timescale to generate a hypothetical business scenario;
a fifth module adapted to create an archive of scenarios comprising data relating to personalised medicine, said data being acquired from real-world case studies;
a sixth module adapted to compare each of the scenarios in the archive with the hypothetical business scenario to identify a first archived scenario that most closely matches the hypothetical business scenario; and
a seventh module adapted to extract information from the first archived scenario, said information being used to justify the intended personalised medicine business plan and provide a one or more guidance parameters on how to implement the intended personalised medicine business plan and thereby develop the intended personalised medicine business plan.

49. A database comprising data formatted in a manner that complies with the system of claim 25.

50. A database comprising data formatted in a manner that complies with the system of claim 47.

51. A database comprising data formatted in a manner that complies with the system of claim 48.
